(19) United States
(12) Patent Application Publication
(10) Pub. No.: US 2026/0306724 A1
(43) Pub. Date: Oct. 8, 2026

(54) Storage Device for a Breast Pump

(71) Applicant: Guocai Rao, Guangdong (CN)

(72) Inventor: Guocai Rao, Guangdong (CN)

(21) Appl. No.: 19/632,403

(22) Filed: Mar. 29, 2026

(30) Foreign Application Priority Data
Apr. 2, 2025 (CN) 202520617451.3

(51) Int. Cl. A61M 1/06

(52) U.S. Cl. CPC A61M 1/062; A61M 1/069; A61M 2205/35; A61M 2205/3673; A61M 2205/8206; A61M 2205/8237; A61M 2209/06; A61M 2209/08

(57) ABSTRACT

The present invention provides a storage device for a breast pump. The storage device comprises a housing having a front face, and a protective cover that is removably connected to the front face of the housing and cooperates with the housing to form an enclosed receiving chamber. A breast pump provided with a breast shield is placed within the receiving chamber. A heating mechanism is disposed on the housing at a position corresponding to the breast shield so that, when the breast pump is received in the receiving chamber, the heating mechanism heats the breast shield by thermal conduction through the housing. Compared with the prior art, the present application utilizes the housing and the protective cover to form the receiving chamber, and uses the receiving chamber to house and store the breast pump, thereby preventing the breast pump from directly contacting an external environment and from being accidentally knocked to the ground and damaged. At the same time, the heating mechanism is capable of heating and thermally insulating the breast shield to bring the breast shield to a temperature approximating a human breast temperature at the time of use, thereby improving user comfort and overall user experience.

### TECHNICAL FIELD

The present invention relates generally to the technical field of breast pumps, and more particularly to a storage device for a breast pump.

### BACKGROUND OF THE INVENTION

A breast pump is an auxiliary device that assists postpartum mothers in expressing breast milk. In the prior art, a breast pump is often placed directly on a tabletop or other exposed surface for storage, which causes the breast pump to be directly exposed to the external environment, making it susceptible to contamination by bacteria and increasing the risk that the breast pump may be accidentally knocked to the ground and damaged. In addition, when a breast pump is used in winter, a breast pump or breast shield that is exposed to the ambient air and remains cold can come into direct contact with a user's breast, which degrades comfort and adversely affects the user experience of postpartum mothers.

### SUMMARY OF THE INVENTION

The purpose of the present utility model is to provide a storage device for a breast pump, so as to improve the problems of improper storage of breast pumps and poor user experience in the prior art.

To achieve the above purpose, the technical solutions of the present utility model are as follows: The present utility model provides a storage device for a breast pump, which comprises:
a housing;
a protective cover, which is detachably connected to a front face of the housing and, together with the housing, forms a receiving chamber;
a breast pump, which is placed in the receiving chamber, the breast pump being provided with a breast shield; and
a heating mechanism, which is disposed on the housing and arranged corresponding to the breast shield.

Compared with the prior art, the present application uses the housing and the protective cover in combination to form the receiving chamber, and uses the receiving chamber to accommodate and store the breast pump, so as to avoid the breast pump directly contacting an external environment and to prevent the breast pump from being knocked to the ground and damaged. At the same time, the heating mechanism can heat and keep the breast shield warm, so that, during use, the temperature of the breast shield can conform to the temperature of a human breast, thereby improving user experience.

In one embodiment, the housing is made of a soft material; the heating mechanism comprises a heating wire or a heating sheet; and the heating mechanism is disposed inside the housing and is not exposed on a front face of the housing.

In one embodiment, a wall-mounting mechanism is provided on a back side of the housing.

In one embodiment, a support cradle is provided on the front face of the housing, and the support cradle is provided with a contoured structure matching the breast pump; when the breast pump is placed on the support cradle, the support cradle is electrically connected to the breast pump through a charging terminal.

In one embodiment, the breast shield comprises an upper shield zone, a lower shield zone, and a middle shield zone, wherein the lower shield zone is arranged corresponding to a milk collection region of the breast pump; the heating mechanism comprises a first heating zone, a second heating zone, and a third heating zone, wherein the first heating zone is arranged corresponding to the upper shield zone, the second heating zone is arranged corresponding to the lower shield zone, and the third heating zone is arranged corresponding to the middle shield zone.

In one embodiment, the storage device for the breast pump further comprises a sterilization mechanism, the sterilization mechanism comprising a UV germicidal lamp; the sterilization mechanism is disposed in the receiving chamber.

The present utility model further provides a storage device for a breast pump, which comprises: a housing; a protective cover, which is detachably connected to a front face of the housing and, together with the housing, forms a receiving chamber; and a breast pump, which is placed in the receiving chamber, the breast pump being provided with a breast shield, and a heating mechanism being provided on the breast shield.

Compared with the prior art, the present application uses the housing and the protective cover in combination to form the receiving chamber, and uses the receiving chamber to accommodate and store the breast pump, so as to avoid the breast pump directly contacting an external environment and to prevent the breast pump from being knocked to the ground and damaged; at the same time, the heating mechanism can heat and keep the breast shield warm, so that, during use, the temperature of the breast shield can conform to the temperature of a human breast, thereby improving user experience.

In one embodiment, a wall-mounting mechanism is provided on a back side of the housing.

In one embodiment, the storage device for the breast pump further comprises a sterilization mechanism, the sterilization mechanism comprising a UV germicidal lamp; the sterilization mechanism is disposed in the receiving chamber.

For better understanding and implementation, the present utility model will be described in detail below with reference to the accompanying drawings.

### BRIEF DESCRIPTION OF THE DRAWINGS

FIG. 1 is a perspective view of the storage device for a breast pump according to the present utility model.

FIG. 2 is a perspective view of the storage device for a breast pump according to the present utility model (with the protective cover omitted).

FIG. 3 is a cross-sectional view of the storage device for an embodiment of the breast pump according to the present utility model.

FIG. 4 is a cross-sectional view of the storage device for an embodiment of the breast pump according to the present utility model.

In the drawings: 10—housing; 101—receiving chamber; 110—wall—mounting mechanism; 120—support cradle; 20—protective cover; 30—breast pump; 310—breast shield; 311—upper shield zone; 312—lower shield zone; 313—middle shield zone; 314—milk collection region; 40—heating mechanism; 410—first heating zone; 420—second heating zone; 430—third heating zone; 50—sterilization mechanism.

### DETAILED DESCRIPTION OF THE INVENTION

To better illustrate the present utility model, the present utility model will be further described in detail below with reference to the accompanying drawings.

It should be understood that the described embodiments are merely some of the embodiments of the present application, and not all of the embodiments. Based on the embodiments in the present application, all other embodiments obtained by those of ordinary skill in the art without creative effort shall fall within the scope of protection of the embodiments of the present application.

The terms used in the embodiments of the present application are for the purpose of describing particular embodiments only, and are not intended to limit the embodiments of the present application. The singular forms “a”, “said” and “the” used in the embodiments of the present application and the appended claims are also intended to include plural forms, unless the context clearly indicates otherwise. It should also be understood that the term “and/or” used herein refers to and includes any and all possible combinations of one or more of the associated listed items.

When the following description refers to the accompanying drawings, unless otherwise indicated, the same numerals in different drawings represent the same or similar elements. The implementations described in the following exemplary embodiments do not represent all implementations consistent with the present application. Rather, they are merely examples of apparatus and methods consistent with some aspects of the present application as detailed in the appended claims. In the description of the present application, it should be understood that terms such as “first”, “second”, “third” etc. are used merely to distinguish similar objects, and are not necessarily used to describe a specific order or sequence, nor are they to be understood as indicating or implying relative importance. For those of ordinary skill in the art, the specific meanings of the above terms in the present application can be understood according to specific circumstances.

In addition, in the description of the present application, unless otherwise specified, “plurality” refers to two or more. “And/or” describes the association relationship of associated objects, indicating that there can be three relationships, for example, A and/or B can represent: A existing alone, A and B existing together, or B existing alone. The character “/” generally indicates that the preceding and following associated objects are in an “or” relationship.

### Embodiment 1

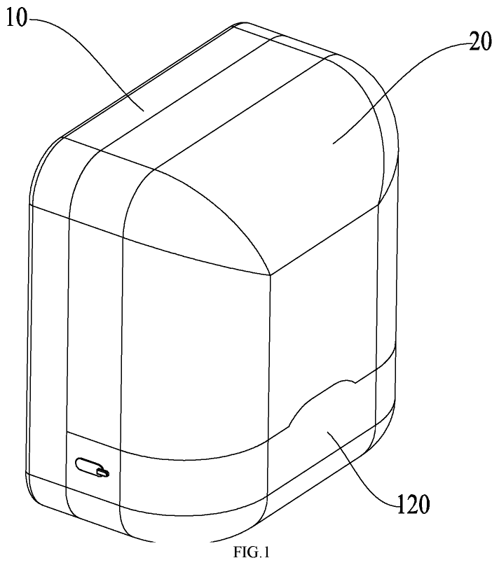

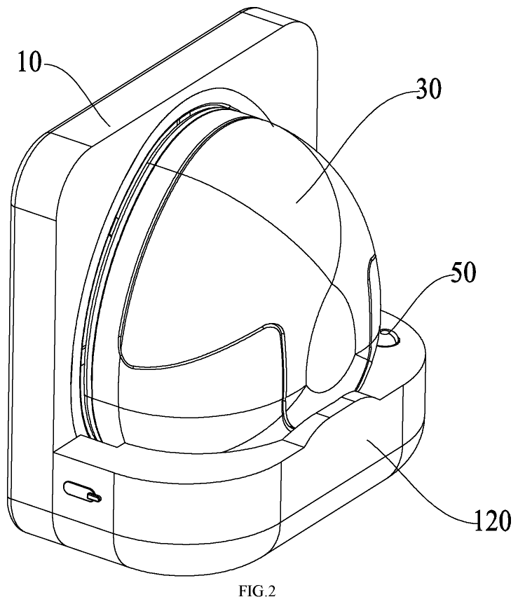

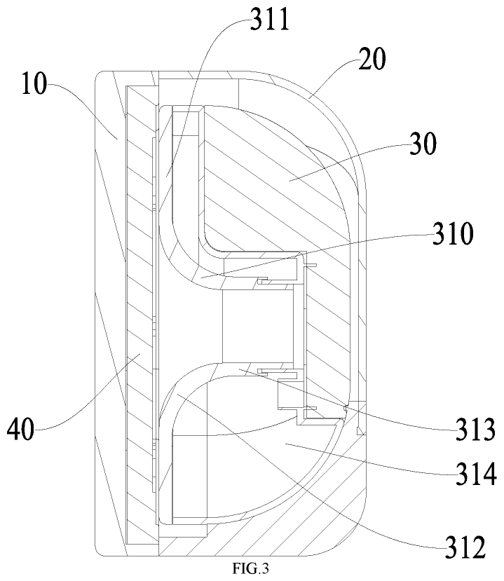

As shown in FIG. 1 to FIG. 3, this embodiment provides a storage device for a breast pump, comprising: housing 10; protective cover 20, which is removably connected to a front face of the housing 10 and cooperates with the housing 10 to form receiving chamber 101; breast pump 30, which is placed in the receiving chamber 101, the breast pump 30 being provided with breast shield 310; heating mechanism 40, which is disposed on the housing 10 and arranged corresponding to the breast shield 310.

Specifically, a user places breast pump 30 in receiving chamber 101, heating mechanism 40 is energized to work and generate heat, the heat is transferred to breast shield 310 and heats and keeps breast shield 310 warm, wherein after the temperature on breast shield 310 is heated to a preset temperature, the temperature is maintained stable to avoid excessive heating that could burn the user. It should be noted that the preset temperature may be preset by the manufacturer or may be set by the user according to their own needs. After preheating is completed, the user only needs to open protective cover 20 for direct use, and during use, since the temperature of breast shield 310 is close to the temperature of a human breast, the user will not feel discomfort, and compared with the cold breast shield 310 of the prior art, breast pump 30 of this embodiment can effectively improve user experience.

In addition, when breast pump 30 is not in use, it is placed in receiving chamber 101, and in addition to receiving chamber 101 providing a storage space for breast pump 30, protective cover 20 and housing 10 protect breast pump 30 to prevent breast pump 30 from being damaged by falling.

Further, when a postpartum mother has issues such as engorgement, heating mechanism 40 can increase heating power according to the user's mode selection to raise the temperature of breast shield 310, thereby achieving a hot compress function to promote blood circulation and milk secretion in the breast and clear the mammary glands.

Compared with the prior art, the present application uses housing 10 and protective cover 20 in combination to form receiving chamber 101, and uses receiving chamber 101 to accommodate and store breast pump 30, avoiding breast pump 30 directly contacting the external environment and preventing breast pump 30 from being knocked to the ground and damaged; at the same time, heating mechanism 40 can heat and keep breast shield 310 warm so that during use it conforms to human breast temperature, improving user experience.

In addition, since this embodiment heats the breast shield through the heating mechanism on the housing, after the breast pump is removed from the receiving chamber it is applied to the breast, allowing the postpartum mother to feel warmth close to body temperature upon initial contact, while also avoiding discomfort from continuous heating. Moreover, since the heating mechanism is not provided on the breast shield, compared with prior art breast shield heating, this embodiment enables the breast shield to be thinner and better conform to the breast.

In this embodiment, housing 10 is made of a soft material, such as silicone, rubber or other soft materials; heating mechanism 40 includes a heating wire or heating pad; heating mechanism 40 is disposed within housing 10 and is not exposed on the front face of housing 10, so that the user will not see the heating wire or heating pad on the front face of housing 10 during use, enhancing the aesthetic appearance of the product. Because housing 10 is made of materials such as silicone, when housing 10 of this embodiment is mounted against a wall, housing 10 can better conform to the wall. When housing 10 of this instance is placed on a tabletop, the friction between silicone housing 10 and the tabletop is greater than that of other hard materials (such as plastic, metal), making the storage device for the breast pump less likely to slide on the tabletop and fall to the ground.

Specifically, a plurality of channels may be provided on the back face of silicone housing 10, heating wires are arranged in the channels as needed, and finally the heating wires in the channels are encapsulated with sealing compound. After the heating wires are energized to generate heat, the heat is transferred to housing 10, and then transferred from the front face of housing 10 to breast shield 310, thereby heating breast shield 310.

In this embodiment, back face of housing 10 is provided with wall-mounting mechanism 110 to mount the storage device for the breast pump of this embodiment against a wall. Specifically, when the storage device for the breast pump of this embodiment is mounted on the wall, the user only needs to place breast pump 30 in receiving chamber 101 during use, and directly remove breast pump 30 from receiving chamber 101 when using.

Further, wall-mounting mechanism 110 may adopt hanging apertures formed in the back face of housing 10, which cooperate with hooks fixedly mounted on the wall. It should be noted that the back face of housing 10 is divided into a first region and a second region according to function, wherein the first region is provided with heating mechanism 40 corresponding thereto, and the second region is provided with the hanging aperture corresponding thereto, avoiding interference between the hanging aperture and heating mechanism 40.

In other embodiments, wall-mounting mechanism 110 may also be a plurality of bolts, with a plurality of mounting holes provided at edges of housing 10, and the bolts pass through the mounting holes to mount housing 10 to the wall, making housing 10 conform to the wall.

In this embodiment, front face of housing 10 is provided with support cradle 120, and support cradle 120 is provided with a contoured structure matching breast pump 30; when breast pump 30 is placed on support cradle 120, support cradle 120 is electrically connected to breast pump 30 through a charging terminal. Specifically, when the storage device for the breast pump of this embodiment is mounted on the wall, support cradle 120 can mount and support breast pump 30, and support cradle 120 is provided with a contoured structure that can better conform to the surface of breast pump 30 to prevent loosening.

Further, protective cover 20 is provided above support cradle 120, and protective cover 20 cooperates with support cradle 120 via a snap-fit structure or magnetic attraction structure, so that protective cover 20 can be fixed on support cradle 120.

Further, support cradle 120 is provided with a control circuit board, a power terminal, and a charging terminal; when breast pump 30 is placed on support cradle 120, support cradle 120 is electrically connected to breast pump 30 through the charging terminal to charge breast pump 30 using the storage device for the breast pump.

Preferably, the charging terminal may be Type-C charging, pogo-pin charging, or wireless charging.

Preferably, the power terminal may be a Type-C interface, and a power cord is connected through the Type-C interface for power input to supply power to breast pump 30 and heating mechanism 40.

Preferably, a battery is provided within support cradle 120 to store electrical energy input by the power terminal, so that the battery can be used for power supply when there is no external power input.

Further, a wireless communication module is provided on the control circuit board, which communicates with a user's mobile device (such as a mobile phone, tablet computer, smart watch, etc.) via Wi-Fi, Bluetooth, etc., so that the user can receive status information of the storage device for the breast pump through the mobile device, and send command information to the storage device for the breast pump.

This embodiment uses support cradle 120 to support breast pump 30, and also uses the space within support cradle 120 to accommodate the control circuit board, battery, etc., rationally utilizing space.

In other embodiments, buttons may also be provided on the surface of support cradle 120, so that the user can select the working mode and working temperature of the storage device for the breast pump through the buttons.

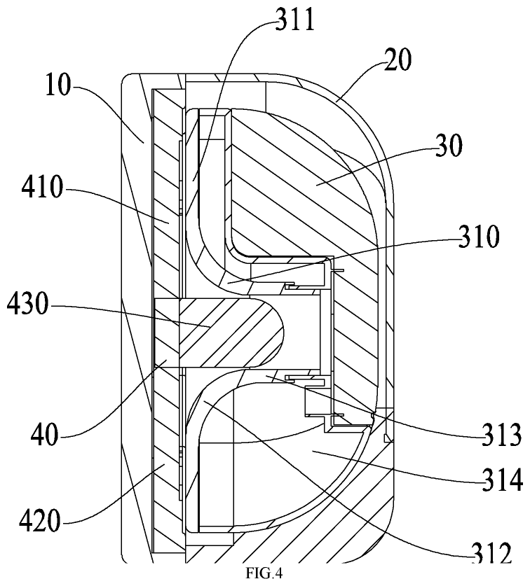

As shown in FIG. 4, to heat breast shield 310 more quickly, in this embodiment, breast shield 310 includes upper shield zone 311, lower shield zone 312, and middle shield zone 313, wherein lower shield zone 312 is arranged corresponding to milk collection region 314 of breast pump 30; heating mechanism 40 includes first heating zone 410, second heating zone 420, and third heating zone 430, wherein first heating zone 410 is arranged corresponding to upper shield zone 311, second heating zone 420 is arranged corresponding to lower shield zone 312, and third heating zone 430 is arranged corresponding to middle shield zone 313 and protrudes toward breast shield 310.

Specifically, the middle position of breast shield 310 (i.e., middle shield zone 313) is spaced away from housing 10, therefore the distance from middle shield zone 313 to heating mechanism 40 is the greatest, and middle shield zone 313 does not directly contact housing 10, whereas upper shield zone 311 and lower shield zone 312 directly contact housing 10. If heated at the same power, the heating speed of middle shield zone 313 is the slowest, because heat from upper shield zone 311 and lower shield zone 312 needs to gradually transfer to middle shield zone 313.

The heating wire of first heating zone 410 of this embodiment heats upper shield zone 311 of breast shield 310 at a first power level, the heating wire of second heating zone 420 heats lower shield zone 312 of breast shield 310 at a first power level, and the heating wire of third heating zone 430 heats middle shield zone 313 of breast shield 310 at a second power level. Wherein the second power level is set lower than the first power level. Third heating zone 430 of this embodiment protrudes toward breast shield 310 to heat middle shield zone 313, cooperating with heat transfer from upper shield zone 311 and lower shield zone 312 to middle shield zone 313, shortening heating time. Moreover, the heating power of third heating zone 430 is lower than that of first heating zone 410 and second heating zone 420 to avoid excessive heating of middle shield zone 313.

In addition, after a postpartum mother uses the breast pump, milk remains in milk collection region 314, and milk collection region 314 corresponds to lower shield zone 312. Generally, milk in breast pump 30 is poured into a special container and refrigerated in the refrigerator. In some usage scenarios, milk from breast pump 30 is fed to the infant ten or more minutes after pumping to ensure the milk is kept warm. After the user finishes using breast pump 30 and cleans the surface of breast shield 310, it is placed back into receiving chamber 101. At this time, the heating wires of first heating zone 410 and third heating zone 430 do not operate, and the heating wire of second heating zone 420 can independently heat lower shield zone 312 of breast shield 310 to keep the milk in milk collection region 314 warm. Until ten or more minutes later, breast pump 30 is removed and the milk from breast pump 30 is poured out to feed the infant. It should be noted that if the time between pumping and feeding the infant is relatively long, it is recommended to remove and refrigerate the milk as early as possible.

In this embodiment, the storage device for the breast pump further includes sterilization mechanism 50, sterilization mechanism 50 comprising a UV germicidal lamp; sterilization mechanism 50 is disposed in receiving chamber 101, using the UV germicidal lamp to sterilize breast pump 30 and the interior of receiving chamber 101.

### Embodiment 2

The difference between this embodiment and Embodiment 1 is that this embodiment further provides a storage device for a breast pump, comprising: housing 10; protective cover 20, which is removably connected to a front face of housing 10 and cooperates with housing 10 to form receiving chamber 101; breast pump 30, which is placed in receiving chamber 101, breast pump 30 being provided with breast shield 310, and heating mechanism 40 being provided on breast shield 310.

Compared with the prior art, the present application uses housing 10 and protective cover 20 in combination to form receiving chamber 101, and uses receiving chamber 101 to accommodate and store breast pump 30, avoiding breast pump 30 directly contacting the external environment and preventing breast pump 30 from being knocked to the ground and damaged; at the same time, heating mechanism 40 of breast shield 310 can heat and keep breast shield 310 warm so that during use it conforms to human breast temperature, improving user experience. When breast pump 30 is not in use, it is placed in receiving chamber 101, and in addition to receiving chamber 101 providing a storage space for breast pump 30, protective cover 20 and housing 10 protect breast pump 30 to prevent breast pump 30 from being damaged by falling.

In one embodiment, back face of housing 10 is provided with wall-mounting mechanism 110.

In one embodiment, the storage device for the breast pump further includes sterilization mechanism 50, sterilization mechanism 50 comprising a UV germicidal lamp; sterilization mechanism 50 is disposed in receiving chamber 101.

Based on the disclosure and teachings of the above description, those skilled in the art to which the present utility model pertains can also make changes and modifications to the above embodiments. Therefore, the present utility model is not limited to the specific embodiments disclosed and described above, and some modifications and variations of the present utility model shall also fall within the scope of protection of the claims of the present utility model. In addition, although some specific terms are used in this specification, these terms are merely for convenience of description and do not constitute any limitation on the present utility model.

1. A storage device for a breast pump, comprising:
a housing having a front face;
a protective cover removably attached to the front face of the housing, the protective cover and the housing together defining an enclosed receiving chamber sized to receive a breast pump therein;
a heating mechanism disposed on the housing and positioned to face and thermally contact a breast shield of the breast pump when the breast pump is received within the receiving chamber, wherein the heating mechanism is configured to heat the breast shield by thermal conduction through the housing.

2. The storage device of claim 1, wherein:
the housing is formed of a soft, flexible material;
the heating mechanism comprises at least one of a heating wire and a heating pad embedded within the housing; and
the heating mechanism is concealed within the housing such that it is not exposed on the front face of the housing.

3. The storage device of claim 2, wherein the soft, flexible material comprises silicone or rubber.

4. The storage device of claim 1, further comprising a wall-mounting mechanism disposed on a rear face of the housing, the wall-mounting mechanism configured to mount the storage device on a wall surface.

5. The storage device of claim 4, wherein the wall-mounting mechanism comprises at least one hanging aperture formed in the rear face of the housing, the hanging aperture configured to engage a hook fixed to the wall surface, and wherein the hanging aperture is disposed in a region of the rear face that does not overlap with the heating mechanism to avoid interference therewith.

6. The storage device of claim 1, wherein the front face of the housing includes a support cradle having a contoured profile shaped to conform to an exterior surface of the breast pump, and wherein the support cradle includes a charging terminal that electrically connects to the breast pump when the breast pump is seated on the support cradle, thereby charging the breast pump.

7. The storage device of claim 6, wherein the charging terminal comprises at least one of a USB Type-C connector, a pogo-pin connector, and a wireless charging coil.

8. The storage device of claim 1, wherein:
the breast shield defines an upper shield zone, a lower shield zone, and a middle shield zone, the lower shield zone corresponding to a milk collection region of the breast pump; and
the heating mechanism comprises a first heating zone aligned with the upper shield zone, a second heating zone aligned with the lower shield zone, and a third heating zone aligned with the middle shield zone.

9. The storage device of claim 8, wherein:
the third heating zone protrudes toward the breast shield relative to the first heating zone and the second heating zone; and
the third heating zone operates at a second heating power level that is lower than a first heating power level at which the first heating zone and the second heating zone operate, thereby preventing overheating of the middle shield zone while compensating for the greater distance between the middle shield zone and the housing.

10. The storage device of claim 8, wherein the second heating zone is independently operable to selectively heat only the lower shield zone corresponding to the milk collection region, thereby maintaining a temperature of milk stored in the milk collection region while the first heating zone and the third heating zone remain inactive.

11. The storage device of claim 1, further comprising a sterilization mechanism disposed within the receiving chamber, the sterilization mechanism comprising at least one ultraviolet (UV) germicidal lamp configured to irradiate interior surfaces of the receiving chamber and the breast pump.

12. The storage device of claim 1, further comprising:
a control circuit board housed within the housing;
a rechargeable battery housed within the housing and electrically connected to the control circuit board; and
a wireless communication module on the control circuit board configured to communicate with a mobile device via at least one of Wi-Fi and Bluetooth, enabling a user to receive status information from the storage device and to transmit operating instructions to the storage device via the mobile device.

13. The storage device of claim 1, wherein the heating mechanism is controllable between:
a warming mode in which the heating mechanism heats the breast shield to a preset temperature approximating human body temperature to warm the breast shield before use; and
a hot-compress mode in which the heating mechanism operates at an elevated power level above the warming mode to promote blood circulation and milk secretion.

14. A storage device for a breast pump, comprising:
a housing;
a protective cover removably attached to a front face of the housing, the protective cover cooperating with the housing to define a receiving chamber configured to store a breast pump having a breast shield;
a heating mechanism integrated into the housing, wherein the heating mechanism is positioned on the housing at a location corresponding to the breast shield of the breast pump when stored in the receiving chamber, such that heat generated by the heating mechanism is transferred through the housing to the breast shield by conduction, wherein the heating mechanism is separate from the breast pump and is not integrated into the breast shield.

15. The storage device of claim 14, wherein the housing is a silicone body having a rear face formed with one or more channels, and wherein the heating mechanism comprises a heating wire routed through the channels and encapsulated by a sealing compound, the heating wire transferring heat through the silicone body to the front face of the housing in thermal contact with the breast shield.

16. The storage device of claim 14, wherein the protective cover is removably attached to the housing by at least one of a snap-fit structure and a magnetic attraction structure.

17. The storage device of claim 14, further comprising:
a wall-mounting mechanism on a rear face of the housing;
a support cradle on the front face of the housing shaped to conform to and support the breast pump;
a sterilization mechanism comprising a UV germicidal lamp disposed within the receiving chamber; and
wherein the heating mechanism comprises a first zone, a second zone, and a third zone respectively aligned with an upper region, a lower region, and a middle region of the breast shield.

18. A method of storing and preheating a breast pump, comprising:
placing a breast pump having a breast shield into a receiving chamber defined between a housing and a protective cover removably attached to a front face of the housing;
activating a heating mechanism disposed within the housing at a position corresponding to the breast shield, the heating mechanism heating the breast shield by thermal conduction through the housing to a predetermined temperature approximating human body temperature; and
removing the protective cover and withdrawing the preheated breast pump from the receiving chamber for immediate use,
wherein the breast shield is heated by the heating mechanism located in the housing and not by any heating mechanism carried by the breast pump itself.

19. The method of claim 18, further comprising:
selecting a hot-compress mode in which the heating mechanism operates at a power level higher than the predetermined temperature to deliver therapeutic heat to promote milk flow before pumping; and
after pumping, returning the breast pump to the receiving chamber and activating a milk-warming sub-mode in which only a second heating zone of the heating mechanism heats a lower region of the breast shield corresponding to a milk collection reservoir, thereby maintaining a temperature of collected milk until feeding.

20. The method of claim 18, further comprising activating a UV germicidal lamp disposed within the receiving chamber to sterilize at least the breast shield and interior surfaces of the receiving chamber while the breast pump is stored therein.
